(19) United States
(12) Patent Application Publication
(10) Pub. No.: US 2008/0286370 A1
(43) Pub. Date: Nov. 20, 2008

(54) Nanoscale Particles Used as Contrasting Agents in Magnetic Resonance Imaging

(76) Inventors: Armin Kuebelbeck, Bensheim (DE); Heike Schilke, Babenhausen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/093,187

(22) PCT Filed: Oct. 17, 2006
(86) PCT No.: PCT/EP2006/009982

§ 371 Date: May 9, 2008

(30) Foreign Application Priority Data
Nov. 10, 2005 (DE) 10 2005 053 618.2

(51) Int. Cl. A61K 9/16; A61K 38/43; A61K 38/16; A61K 39/395; A61K 49/18

(52) U.S. Cl. 424/490; 424/489; 424/94.1; 514/2; 424/130.1; 424/9.3

(57) ABSTRACT

The invention relates to nanoscale particles as contrast agents for magnetic resonance imaging, consisting of a core having an inert matrix, one or more covalently bonded organic complexing agents in which one or more metal ions having unpaired electrons are bonded, and optionally one or more biomolecules covalently bonded to the surface of the cores, and to a process for the production of these nanoparticles.

The invention relates to nanoscale particles which consist of a core having an inert matrix, one or more covalently bonded organic complexing agents in which one or more metal ions having unpaired electrons are bonded, and optionally one or more biomolecules covalently bonded to the surface of the cores, and to a process for the production thereof.

Owing to its optimal signal transduction (T1 shortening, strong paramagnetism due to 7 unpaired electrons), gadolinium (Gd) is employed in MRI (magnetic resonance imaging). Due to the 7 unpaired electron pairs, the gadolinium induces a strong electromagnetic alternating field which influences the spin of the adjacent water protons in such a way that their relaxation time is reduced.

Intravenously administered solutions of gadolinium salts have an acutely toxic action. The toxicity affects, inter alia, the smooth and striated muscles, the function of the mitochondria and blood clotting. It has therefore been attempted to find ways of reducing the toxicity of this metal without impairing its paramagnetic properties—i.e. the tendency to migrate into magnetic fields. The best way to this aim, which has also resulted in the commercial production of Gd-containing contrast agents, is chelation.

To this end, complexing agents having very high complex formation constants are employed. Examples of these complexing agents are DOTA and DTPA.

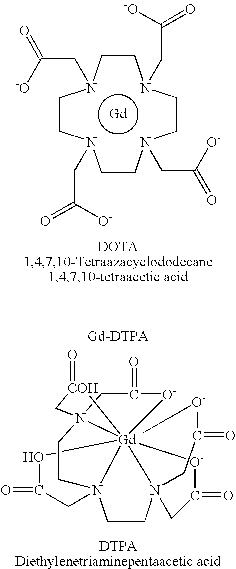

The most stable commercial gadolinium chelate complex to date is macrocyclic gadoteric acid (Gd-DOTA; commercially available, for example, as DOTAREM®, Guerbet). The risk of dissociation and thus of liberation of toxic gadolinium ions (LD₅₀ about 0.1 mmol kg⁻¹) on use of gadoteric acid as MRI contrast agent is very low. In contrast to other complexes, whose release half-life stability in acidic gastric juice (measured in 0.1 molar HCl solution as standardised model) is in the range from seconds to hours, gadoteric acid here has a half life of more than one month. Exchange of gadolinium with endogenic metal ions, such as copper or zinc, is also significantly less than 1%, while it can be up to 35% in other complexes.

Gd is completely surrounded by the organic acid DOTA and sits in the centre of the chelate molecule like in a cave, as shown by X-ray crystallographic studies. The toxicity of gadolinium is thus masked virtually completely, while its paramagnetic properties, which make it interesting as MRI contrast agent, are retained.

The use of contrast agents in an MRI increases the informative value of the display of organs. In the normal case, 10 to 15 ml (0.2 ml/kg of body weight) of contrast agent are injected intravenously. The Gd-containing contrast agent used results in a shortening of the relaxation time and thus in a stronger signal in the images produced.

Contrast agents based on the transition elements manganese, iron or copper are only used in the case of specific questions, in particular relating to the liver.

Paramagnetic complexes, such as gadoteric acid, have hydrophilic properties and do not pass through the blood/brain barrier. After intravenous injection, rapid vascular distribution occurs, followed by interstitial distribution; a preference for a certain organ is not observed. The complexes are excreted in unchanged form via the kidneys within a few hours by means of glomerular filtration. Gadoteric acid is eliminated to the extent of 75% after three hours. The pharmacokinetics described make Gd a contrast agent which is especially suitable for the diagnosis of movements of extracellular fluid, as occurs in the case of tumours, oedemas, necroses and ischaemias.

Gd-DOTA is very well tolerated. Thus, two studies with more than 5000 patients have shown that the side-effect rate is between 0.84% and 0.97%. Of 4169 patients in the larger of the two studies (Caillé 1991), only 8 suffered from nausea and 5 from vomiting, which gives a total rate of 0.31% for these side effects. Temperature, headaches, an unwell feeling, skin rash and an unpleasant taste in the mouth occurred in less than 0.15% of all patients. Systemic osmolality effects are also negligible in the case of gadoteric acid. The only organ in which increased concentrations of Gd-DOTA are evident is the kidney, which is probably due to pharmacokinetic reasons. However, tolerance studies with renal insufficiency patients whose creatinin clearance was less than 60 ml/min showed absolutely no adverse effect of Gd-DOTA on vital parameters or renal function. In contrast to Gd-DTPA-BMA, Gd-DOTA has not caused pseudohypocalcaemia. The recommendation in the case of renal insufficiency: monitoring+take all measures for preventing renal insufficiency (hydration, dose restriction, risk/benefit assessment). For all these reasons, gadoteric acid is approved as MRI contrast agent not only for adults, but also for children and infants.

Nanoscale particles containing gadolinium(III) have been known for some years and have advantages over individual complexed gadolinium(III) ions which are significant in the application as contrast agent in diagnostics:
The accumulation of a multiplicity of complexed gadolinium(III) ions on a sphere surface results in a significantly stronger signal in the magnetic resonance image compared with individual gadolinium complexes. This enables either the dose of contrast agent to be reduced or alternatively a stronger signal to be obtained at the same dose through the improved signal/noise ratio.

C. Platas-Iglesias et al., Chem. Eur. J. 2002, 8, No. 22, 5121-5131 “Zeolite GdNaY nanoparticles with very high relaxivity for application as contrast agents in magnetic resonance imaging”, disclose Gd³⁺-loaded zeolite NaY nanoparticles in which gadolinium is only bonded via Coulomb forces, i.e. not covalently. Since the pore size of the Y zeolites here is only 1.3 nm, free proton exchange with the surrounding tissue is strongly hindered.

WO 00/30688 (Bracco) describes substituted polycarboxylate ligand molecules and corresponding metal complexes, such as Gd-DTPA and Gd-DOTA derivatives, as contrast agents for MRI.

WO 2004/009134 (Bracco) describes Gd chelate complexes as MRI contrast agents which are surrounded by the cell.

WO 96/09840 (Nycomed) describes a diagnostic agent comprising a particulate material whose particles comprise a diagnostically active, essentially water-insoluble crystalline material of a metal oxide (iron oxide) and a poly-ionic coating agent (for example chitosan, hyaluronic acid, chondroitin).

WO 04/083902 (Georgia Tech Research Corp.) describes magnetic nanoparticles (for example Gd chelates) having a biocompatible coating (for example phospholipid-polyethylene glycol) which may carry biomolecules, such as nucleic acids, antibodies, etc.

WO 03/082105 (Barnes Jewish Hospital) describes Gd-DTPA-PE and Gd-DTPA-BOA chelate complexes which are surrounded by a lipid/surfactant coating.

The object of the present invention was to prepare novel contrast agents which avoid the disadvantages of the compounds mentioned above.

This has been achieved, surprisingly, by the use of high-purity silicon dioxide as support of the covalently bonded lanthanide complexes (preferably gadolinium complexes). Silicon dioxide is extremely well tolerated in the patient's body and is thus far superior to many other materials from the prior art. Examples in this respect are given, inter alia, by: Jain, T. K.; Roy, I.; Dee, T. K.; Maitra, A. N., J. Am. Chem. Soc. 1998, 120, 11092-11095, Shimada, M.; Shoji, N.; Takahashi, A., Anticancer Res. 1995, 15, 109-115 and Lal, M.; Levy, L.; Kim, K. S.; He, G. S. Wang, X.; Min, Y. H.; Pakatchi, S.; Prasad, P.N., Chem. Mater. 2000, 12, 2632-2639.

The present invention thus relates to nanoscale particles consisting of a core having an inert matrix, one or more covalently bonded organic complexing agents in which a metal ion having unpaired electrons is bonded, and optionally one or more biomolecules covalently bonded to the surface of the cores.

The present invention furthermore relates to nanoscale particles consisting of a core having an inert matrix, optionally one or more biomolecules covalently bonded to the surface of the cores, and one or more organic complexing agents which are covalently bonded to the surface of the cores via a linker and in which a metal ion having unpaired electrons is bonded. The core or support having the inert matrix preferably consists of silicon dioxide, titanium dioxide, aluminium oxide or zirconium dioxide. Silicon dioxide is particularly preferred. The monodisperse silicon dioxide particles are produced by known methods (see EP 0216278) by hydrolysis of tetraalkoxysilanes. The average particle diameter of the monodisperse particles here is 10 to 500 nm, preferably 30 to 300 nm. In principle, however, polymers, for example polystyrene lattices, can also be used.

Furthermore, it is possible to coat other nanoparticles with a thin layer of silicon dioxide in a first step. The coating is possible in a simple manner by the sol-gel process known to the person skilled in the art. To this end, nanoparticles are suspended in an ethanolic/aqueous solution, and a silicic ester, for example tetraethyl orthosilicate (TEOS), is added. The hydrolysis of the silicic ester is initiated by the addition of aqueous ammonia solution, if necessary at elevated temperatures. The precipitated silicon dioxide is preferably deposited on the nanoparticles in the suspension. The layer thickness can be set very precisely, for a known amount and known average diameter of the nanoparticles to be coated, through the amount of silicic ester employed.

The coated nanoparticles can be separated off and purified by ultrafiltration or centrifugation at particle diameters >about 50 nm.

The metal ions employed are preferably paramagnetic ions from the lanthanide group. Particular preference is given to the use of gadolinium(III) ions.

The organic complexing agents employed are preferably compounds from the oligo- or polycarboxylate group. Particular preference is given to the use of diethylenetriaminepentaacetic acid (DTPA) or 1,4,7,10-tetraazacyclo-decane-1,4,7,10-tetraacetic acid (DOTA).

The metal chelate complexes are covalently bonded to the surface of the support via a linker, preferably via a silane. A preferred linker is 3-amino-propyltriethoxysilane (APTES).

It is particularly advantageous to prepare the metal chelate complexes via the copper-catalysed dipolar 1,3-cycloaddition of azidene to alkynes, the so-called Huisgen reaction. This reaction, known to the person skilled in the art, gives stable 1,2,3-triazoles under very mild conditions, with excellent yields, and facilitates in a simple manner the synthesis of even very complex molecules. This reaction has therefore recently attracted increased interest again under the term “click chemistry”, which is reflected in a large number of publications (see Bräse et al, Angew. Chem. 2005, 117, 5336; Kolb, Finn and Sharpless, Click Chemistry: Diverse Chemical Function from a Few Good Reactions, Angew. Chem. Int. Ed. 2001, 40, 2004-2021).

Surprisingly, it has been found that the above-mentioned Huisgen reaction can also advantageously be used for the functionalisation of the surface of nanoparticles. This means that “click chemistry” can also be employed for heterogeneous solid-phase reactions on nanoscale particles.

The present invention thus furthermore relates to a process for the production of nanoscale particles comprising the following process steps:
a) production of nanoparticles, preferably from silicon dioxide, titanium dioxide, aluminium oxide and/or zirconium dioxide, by wet-chemical methods
b) coating of the nanoparticles with a monomolecular layer of a halosilane
c) reaction of the nanoparticles with an azide-containing agent to give nanoparticles functionalised with azide groups
d) preparation of one or more organic complexing agents containing one or more amines and one or more polycarboxylic acids, polycarboxylic anhydrides, polycarboxyl chlorides or polycarboxylic esters
e) loading of one or more complexing agents with metal ions from the lanthanide group
f) reaction of the nanoparticles functionalised with azide groups from step c) with the organic complexing agent(s) loaded with metal ions from step e).

The halosilane employed is preferably, for example, 3-(chloropropyl)-triethoxysilane.

The alkynamines employed can be all known alkynamines, preference being given to the use of propargylamine or 6-amino-1-hexyne. This is reacted with a polycarboxylic acid which is suitable for complex formation, a polycarboxylic anhydride, a polycarbonyl chloride or a polycarboxylic ester containing a good leaving group. A carboxamide is synthesised by known processes. As polycarboxylic acids, DOTA and DTPA or derivatives thereof (for example as Li salts) are preferably reacted with a corresponding amine. It is ensured during the reaction that only one carboxylic acid function of the polycarboxylic acid reacts with the amine (1:1 batch). The reaction of, for example, DTPA dianhydride with propargylamine is carried out by the known Schotten-Baumann method.

Biomolecules, such as, for example, enzymes, peptides/proteins, receptor ligands or antibodies, may additionally be covalently bonded to the nanoparticles. The specific coupling thereof to the target tissue in the patient's body simplifies imaging and consequent diagnosis.

The nanoparticles may furthermore be coated with dextran or polyethylene glycol in order to increase the biocompatibility.

The present invention furthermore relates to the use of the nanoscale particles as contrast agents for magnetic resonance imaging. The particles according to the invention can be used as contrast agents in magnetic resonance imaging since the metal ions arranged on the surface are able to interact with the surrounding protons, for example from tissue fluid.

The following examples are intended to explain the present invention in greater detail without restricting it.

### EXAMPLE 1

### Production of Monodisperse Silicon Dioxide Particles Having an Average Particle Diameter of 250 nm with Surface-Bonded Gd(Iii)

### 1.1 Production of Monodisperse Silicon Dioxide Particles

The monodisperse silicon dioxide particles are produced as described in EP 0216278 B1, by hydrolysis of tetraalkoxysilanes in aqueous/alcoholic/ammoniacal medium, where firstly a sol of primary particles is produced, and the resultant SiO₂ particles are subsequently brought to the desired particle size by continuous metered addition of tetraalkoxysilane controlled to the extent of the reaction.

1.2 Functionalisation with 3-Aminopropyltriethoxysilane (APTES)

10 g of the silicon dioxide particles produced in the first step were suspended in 20 ml of 2-propanol. 0.25 ml of APTES, diluted with 5 ml of 2-propanol, was subsequently added dropwise, and the mixture was stirred for 2 hours at 80° C. under a reflux condenser.

The suspension was washed 8 times with 2-propanol with the aid of a centrifuge at 4000 min⁻¹ until APTES was no longer detectable—by means of a drop test with ninhydrin—in the wash solution.

1.3 Amide Formation with Diethylenetriaminepentaacetic Acid (DTPA)

25 ml of dimethyl sulfoxide (DMSO) were added to the silicon dioxide particles functionalised with APTES in the second step, and the 2-propanol was distilled off in vacuo in a rotary evaporator. 0.58 g of diethylene-triaminepentaacetic dianhydride (DTPA-ca) was subsequently added to the suspension, and the mixture was stirred at 150° C. for 2 hours. After cooling to room temperature, the reaction product was poured into 200 ml of 0.1 N TRIS buffer (pH 7.0) and washed a number of times with deionised water in a centrifuge.

1.4 Loading with Gadolinium(III) Ions

0.486 g of anhydrous gadolinium(III) chloride was added to the suspension obtained in the 3rd step, and the mixture was stirred at room temperature for 8 hours. The suspension was subsequently washed with deionised water using a centrifuge until chloride was no longer detectable in the wash water by means of silver nitrate solution. The reaction product was then dried by freeze-drying.

### Characterisation:

The dried, gadolinium-loaded silicon dioxide particles were dissolved in dilute hydrofluoric acid, and the gadolinium content was determined by ICP-MS. 0.13% of gadolinium was found in the sample. A sample of the silicon dioxide particles was again washed intensively (3×) with deionised water and, after drying, re-analysed by ICP-MS. The gadolinium content was determined as 0.14%. The slightly higher gadolinium content can be explained by the different degrees of drying or limitations in the measurement method. However, the crucial factor is that the repeated washing of the silicon dioxide particles did not reduce the gadolinium content, i.e. the gadolinium is quite clearly strongly covalently bonded to the surface of the nonporous silicon dioxide particles. The same result is also obtained in the case of treatment with 1 N hydrochloric acid.

### EXAMPLE 2

### Production of Monodisperse Silicon Dioxide Particles Having an Average Particle Diameter of 90 Nm with Surface-Bonded Gd(III)

### 2.1 Production of the Particles

As described in Example 4 of EP 0216278 B1

### 2.2 Functionalisation of the Particles

10 g of the silicon dioxide particles produced in the first step were suspended in 20 ml of 2-propanol. 0.50 ml of APTES, diluted with 5 ml of 2-propanol, was subsequently added dropwise, and the mixture was stirred for 2 hours at 80° C. under a reflux condenser.

The suspension was washed 8 times with 2-propanol with the aid of a centrifuge at 4000 min⁻¹ until APTES was no longer detectable—by means of a drop test with ninhydrin—in the wash solution.

2.3 Amide Formation with Diethylenetriaminepentaacetic Acid (DTPA)

25 ml of dimethyl sulfoxide (DMSO) were added to the silicon dioxide particles functionalised with APTES in the second step, and the 2-propanol was distilled off in vacuo in a rotary evaporator. 1.0 g of diethylene-triaminepentaacetic dianhydride (DTPA-ca) was subsequently added to the suspension, and the mixture was stirred at 150° C. for 2 hours. After cooling to room temperature, the reaction product was poured into 200 ml of 0.1 N TRIS buffer (pH 7.0) and washed a number of times with deionised water in a centrifuge.

2.4 Loading with Gadolinium(III) Ions

1.0 g of anhydrous gadolinium(III) chloride was added to the suspension obtained in the 3rd step, and the mixture was stirred at room temperature for 8 hours. The suspension was subsequently washed with deionised water using a centrifuge until chloride was no longer detectable in the wash water by means of silver nitrate solution. The reaction product was then dried by freeze-drying.

### Characterisation

The dried, gadolinium-loaded silicon dioxide particles were dissolved in dilute hydrofluoric acid, and the gadolinium content was determined by ICP-MS.

0.2% of gadolinium was found in the sample. The higher Gd content, compared with the particles produced in Example 1, can be explained by the higher surface area to volume ratio of the smaller particles. About 1200 gadolinium ions are calculated to be located on the surface of one of the 90 nm particles.

### EXAMPLE 3

Production of monodisperse silicon dioxide particles having an average particle diameter of 250 nm with surface-bonded Gd(III)

### 3.1 Production of the Silicon Dioxide Nanoparticles

16.7 ml of tetraethyl orthosilicate are added at room temperature to a mixture of 41.5 ml of demineralised water and 111 ml of ethanol, and a homogeneous solution is produced by stirring. 26 ml of 25% by weight ammonia solution are subsequently added, the mixture is stirred vigorously for a further 15 sec. and then left to stand for 1 h. Continuing condensation to give silicon dioxide nanoparticles can be observed from clouding of the solution about 1 min. after addition of the ammonia solution. The reaction mixture is not worked up, but instead fed directly to the next reaction step.

3.2 Reaction with a Halosilane

The nanoparticles produced in the first step are coated with a monomolecular layer of a halosilane. To this end, 80 μl of 3-(chloropropyl)-triethoxysilane are added to the reaction mixture from the 1st step, and the mixture is stirred at 80° C. for 5 h. The particles are subsequently centrifuged off and washed with demineralised water until neutral.

### 3.3 Preparation of the Surface-Bonded Azide

The nanoparticles produced and washed in step 2 are suspended in 50 ml of demineralised water, 66 mg of sodium azide are added, and the mixture is stirred at 50° C. for 24 h. The halogen chlorine is replaced by the pseudohalogen azide by nucleophilic substitution. The azide-containing nanoparticles are separated off from the starting materials in the centrifuge, washed with demineralised water and stored as an aqueous suspension.

3.4 Preparation of the Alkyne (Polycarboxylic Monoalkyneamide) by Reaction of DTPA Dianhydride with Propargylamine (Schotten-Baumann Method)

0.19 g (1 mmol) of 1-ethyl-3-(3-dimethylaminopropyl)carbodiimide hydro-chloride (EDC) from Aldrich and 0.15 g (1.5 mmol) of triethylamine from Merck and 2 ml of dimethylformamide (Merck) are added to 0.62 g (1 mmol) of diethylenetriamine-1,7-tetrakis(t-butyl acetate)-4-acetic acid, Article B-365 from Macrocyclics. After vigorous stirring for 10 min at room temperature, 0.06 g (1 mmol) of propargylamine are added.

The reaction mixture is stirred for a further 8 h at room temperature. The reaction is monitored by thin-layer chromatography. The reaction mixture is taken up in 10 ml of dichloromethane, washed by shaking 3 times with 20 ml of 0.1 molar hydrochloric acid and 3 times with 20 ml of saturated aqueous NaHCO₃. The mixture is finally washed by shaking with saturated aqueous sodium chloride solution and dried over anhydrous sodium sulfate. The dichloromethane is stripped off in a rotary evaporator, and the oily residue is taken up in 4 ml of tetrahydrofuran/ethanol (1:1 by volume). 1 ml of water and 0.1 g (4.4 mmol) of lithium hydroxide are added to the mixture in order to cleave off the ester-protecting groups. The hydrolysis mixture is stirred overnight and evaporated to dryness in a rotary evaporator. The reaction product is taken up in 10 ml of water and adjusted to pH 7 using 1 molar hydrochloric acid.

3.5 Loading of the Complexing Agent with Gadolinium(III) Ions

10 ml of 0.1 molar gadolinium(III) chloride solution (=1 mmol) are added to the solution of the lithium salt of diethylenetriaminepentaacetic 4-propargylamide prepared in step 4, and the mixture is stirred for 30 minutes.

3.6 Production of Nanoparticles Modified with Complexing Agent Molecules (Huisgen Reaction)

The nanoparticle suspension prepared in step 3 and functionalised with azide groups was adjusted to a neutral pH by means of TRIS buffer. The amount of polycarboxylic monoalkyneamide (from step 5) calculated in advance was added dropwise to the nanoparticle suspension in the presence of 50 mg of copper(1) chloride. After stirring for 16 hours at room temperature, the reaction was terminated. The particles were centrifuged off and washed vigorously 3× with 0.1 molar hydrochloric acid and finally with demineralised water.

The gadolinium content of the particles was determined as 0.3% by means of ICP-MS.

1. Nanoscale particles consisting of:
a core having an inert matrix
one or more covalently bonded organic complexing agents in which one or more metal ions having unpaired electrons are bonded and
optionally one or more biomolecules covalently bonded to the surface of the cores.

2. Nanoscale particles consisting of:
a core having an inert matrix
optionally one or more biomolecules covalently bonded to the surface of the cores and
one or more organic complexing agents which are covalently bonded to the surface of the cores via a linker and in which a metal ion having unpaired electrons is bonded.

3. Nanoscale particles according to claim 1, characterised in that the core consists of silicon dioxide, titanium dioxide, aluminium oxide and/or zirconium dioxide.

4. Nanoscale particles according to claim 3, characterised in that the core consists of silicon dioxide.

5. Nanoscale particles according to claim 1, characterised in that they have an average particle diameter of 10 to 500 nm, preferably 30 to 300 nm, and are monodisperse.

6. Nanoscale particles according to claim 1, characterised in that the metal ion is selected from the lanthanide group.

7. Nanoscale particles according to claim 1, characterised in that the metal ion is a gadolinium(III) ion.

8. Nanoscale particles according to claim 1, characterised in that the organic complexing agent is selected from the oligo- or polycarboxylate group.

9. Nanoscale particles according to claim 8, characterised in that the organic complexing agent is diethylenetriaminepentaacetic acid (DTPA) or 1,4,7,10-tetraazacyclododecane-1,4,7,10-tetraacetic acid (DOTA).

10. Nanoscale particles according to claim 1, characterised in that the covalently bonded biomolecules employed are enzymes, peptides/proteins, receptor ligands or antibodies.

11. Nanoscale particles according to claim 2, characterised in that the linker employed is a silane.

12. Nanoscale particles according to claim 11, characterised in that the linker employed is 3-aminopropyltriethoxysilane (APTES).

13. Process for the production of nanoscale particles having the following process steps:
a) production of nanoparticles, preferably from silicon dioxide, titanium dioxide, aluminium oxide and/or zirconium dioxide, by wet-chemical methods
b) coating of the nanoparticles with a monomolecular layer of a halosilane
c) reaction of the nanoparticles with an azide-containing agent to give nanoparticles functionalised with azide groups
d) preparation of one or more organic complexing agents containing one or more amines and one or more polycarboxylic acids, polycarboxylic anhydrides, polycarbonyl chlorides or polycarboxylic esters
e) loading of one or more complexing agents with metal ions from the lanthanide group
f) reaction of the nanoparticles functionalised with azide groups from step c) with the organic complexing agent(s) loaded with metal ions from step e).

14. Process according to claim 13, characterised in that a polycarboxylic monoalkyneamide is prepared in step d) from organic complexing agents, such as 1,4,7,10-tetraazacyclododecane-1,4,7,10-tetraacetic acid (DOTA) or diethylenetriaminepentaacetic acid (DTPA) or derivatives thereof, and a corresponding alkynamine.

15. Process according to claim 13, characterised in that the alkynamine employed in step d) is propargylamine or 6-amino-1-hexyne.

16. Process according to claim 13, characterised in that the metal ions employed in step e) are gadolinium(III) ions.

17. A method of enhancing contrast for magnetic resonance imaging comprising administering nanoscale particles of claim 1.
